(19) United States
(12) Patent Application Publication
(10) Pub. No.: US 2006/0100716 A1
(43) Pub. Date: May 11, 2006

(54) Open-pored metal coating for joint replacement implants and method for production thereof

(76) Inventor: Reto Lerf, Muhen (CH)

Correspondence Address: KNOBBE MARTENS OLSON & BEAR LLP, 2040 MAIN STREET, FOURTEENTH FLOOR, IRVINE, CA, 92614, US

(21) Appl. No.: 10/519,338

(22) PCT Filed: Jun. 27, 2002
(86) PCT No.: PCT/EP03/05586

§ 371 Date: Sep. 19, 2005

(30) Foreign Application Priority Data
Jun. 27, 2002 (DE) 102 28 746.5
Sep. 17, 2002 (DE) 102 43 101.9

(51) Int. Cl. A61F 2/28; B31D 3/00

(52) U.S. Cl. 623/23.5; 216/56; 419/2; 623/23.72

(57) ABSTRACT

The invention relates to a method of producing an open-pored coated joint replacement implant, wherein at least one layer of a biocompatible metal or an alloy thereof is applied to a virgin surface of the implant, to produce an implant surface. A surface micro-structure is then produced on the implant surface. That is carried out by means of etching of the implant surface, for example by means of an acid bath or by means of plasma etching, or by the application of fine biocompatible particles to the implant surface. The layer thickness of the open-pored surface layer is in the range from 0.5 mm to 1.5 mm, the porosity being at least 40%.

The invention relates to an open-pore biocompatible surface layer, to a method of producing an open-pore coated implant, to an implant, and to the use of an open-pore biocompatible surface layer, according to the preambles of patent claims 1, 8, 26 and 27.

Implants, and especially joint replacement implants, are becoming ever more important in restorative and curative medicine. In that context, in the case of cementless joint replacement implants, the mechanically stable anchoring of the implant in the bone is of prime importance and is essential for the long-term stability and tolerability of the implants. Hitherto, however, loosening of the implant has very frequently resulted in osteolysis, caused by abrasion particles. Clinically, this so-called aseptic loosening is the most frequent cause of revision operations, for example on hip joints. Consequently, a reduction in abrasion is an objective in the development of joint replacement implants. A further objective is to prevent abrasion particles from spreading along the implant. In this case, it is of prime importance that the bone is presented with an optimised joint replacement implant surface so that it can grow into the implant. Aseptic loosening is reduced as a result. The surface structure and/or surface coating of a joint replacement implant is/are therefore of crucial importance because they allow—or prevent or impede—bone growth into the implant or into the implant surface.

For the production of such surface layers, porous layers have hitherto been found to be advantageous. Various methods are known by means of which porous layers of such a kind can be produced. Materials used in this instance are biocompatible materials, especially metals such as, for example, titanium. The surface layers are arranged, for the most part, on bone implants so that their long-term anchorage in the bone is improved. The porous layers required can be produced, for example, by means of a sintering technique, the structures and the sintering conditions being so selected that cavities between the metal or titanium particles applied to the surface are preserved.

At this juncture, V. Galante et al., JBJS, 53A (1971), page 101-114, describes, for example, the sintering of a mesh of fine titanium wires onto a substrate. The two U.S. Pat. Nos. 3,855,638 and 5,263,986 disclose a method wherein a titanium powder comprising spherical particles of different sizes is sintered onto a substrate. On the other hand, the U.S. Pat. No. 4,206,516 uses a ground titanium hydride powder comprising angular particles, which again is sintered onto a substrate.

It is furthermore possible by means of sintering to produce a skeleton of titanium from a mixture of thermally unstable position-retainers and titanium powder or of position-retainers and titanium hydride powder. Methods in that regard are described, for example, in the Patent specifications U.S. Pat. No. 5,034,186 or WO 01/19556; likewise, a method of the company Intermedics, Austin, USA entitled “Cancellous Structured Titanium” addresses such a possibility.

However, in all titanium layers produced by a sintering process of such a kind, it is inherently disadvantageous that the roughness of the erstwhile titanium particles or titanium fibres is smoothed out as a result of surface diffusion. Even though the bone can grow into the pores, it is scarcely possible for the bone cells, on a microscopic scale, to gain any hold on the smooth titanium surface. That disadvantage can be avoided by dramatically reducing the time for which the titanium particles are exposed to high temperatures. That is the case in, for example, the flame spraying process. The gain in roughness which can be achieved in this method in the microscopic range is, however, offset by a serious disadvantage in that flame-sprayed or plasma-sprayed titanium layers have virtually no pores that are open to the outside and they consequently prevent ingrowth of the bone per se because the requisite pores are absent. U.S. Pat. No. 4,542,539, for example, has set out to solve that problem. Further articles in that regard are to be found, for example, in AESCULAP, Wissenschaftliche Information 22: “Die PLASMAPORE-Beschichtung fiur die zementlose Verankerung von Gelenkendo-prothesen” [“The PLASMAPORE coating for cementless anchoring of joint endoprostheses”] or under “Osteointegration, Oberflaichen-und Beschichtungen orthopadischer Implantate für den zementfreien Einsatz” [“Osteointegration, surfaces and coatings of orthopaedic implants for cementless use”] of PI Precision Implants AG. However, it has not been possible to solve the previous disadvantages of a flame spraying process of such a kind.

The problem of the titanium surface becoming flattened as a result of the action of high temperatures over a long period can also be avoided by applying heat to the surface only locally and to a limited extent. That can be accomplished, for example, by means of individual spot-welds. The U.S. Pat. No. 5,139,528 discloses, for the purpose, a method wherein a multilayer wire mesh of titanium fibres is “tied” to a substrate. However, it is disadvantageous, in that latter method of reduced heating action, that the layer applied exhibits no actual roughness in the sub-micrometre range. Rather, such a coating, because of the regularity of the multilayer wire mesh of titanium fibres, does not have any macro-roughness, that is to say no peaks and troughs, but only pores which extend downwards away from the surface. In the case of an implant produced in such a manner, it is also not possible, consequently, for bone to grow in effectively.

A further method, which is disclosed in U.S. Pat. No. 5,456,723, achieves a roughness in the sub-micrometre range by etching a titanium surface, which has previously been abrasively blasted. A surface produced in that manner does not, however, have cavities which extend further into the surface and into which the bone could grow.

In accordance with the above, it can be stated in summary that there have hitherto been many different attempts at producing, on a joint replacement implant, a surface which is structured in a satisfactory manner for the ingrowth of bone. It has, however, hitherto been possible merely

to produce open-pore coatings into which the bone can grow but which, in the sub-micrometre range, do not provide any topographical stimuli for osteoblast adhesion and consequently for the ingrowth of bone in a manner which is rapid or better than the prior art;

to make available a surface method which provides a roughness of a few micrometres and a sub-micrometre structure for better adhesion of the osteoblasts, although those surfaces do not have an open pore structure into which the bone could grow;

to produce coatings having a high degree of roughness in the region of a few tens of micrometres, which have a limited degree of porosity, but which again do not have an actual sub-micrometre structure;

to produce open-pore coatings into which the bone can grow and whose surface has sub-micrometre roughness, but which do not have a sufficiently high degree of macro-roughness and which consequently cannot “grab onto” the bone.

The objective of the present invention is accordingly to fill the afore-mentioned deficiency, the problem of the invention being to form the surface of a joint replacement implant so that the surface has stable cavities which are open to the outside and which are of sufficient size for vascularised bone tissue to grown in, the surface having very good biocompatibility, that is to say a bioinert or slightly bioactive property, and also a specifically adjusted degree of sub-micrometre roughness provided anchoring points for the osteoblasts.

The problem is solved by an open-pored biocompatible surface layer according to patent claim 1, by a method of producing a surface of such a kind according to patent claim 8, by an implant according to patent claim 26, and by use according to patent claim 27.

The problem of the invention is especially solved by the provision of an implant, especially a joint replacement implant, which has an open-pored surface layer according to the invention.

Furthermore, the problem is solved by a method of producing an open-pored coated implant, especially a joint replacement implant, which comprises the following steps:

application of at least one layer of a biocompatible metal or an alloy thereof to a virgin surface of the implant, to produce an implant surface,

production of a surface micro-structure on the implant surface by means of etching of the implant surface and/or application of fine biocompatible particles to the implant surface.

A basic central idea of the invention is that there is first produced, as a base, an implant surface which in a second step is provided in controlled manner with a surface micro-structure. This can be accomplished, on the one hand, by proceeding inwards, namely by etching the implant surface. Structuring of the surface can be assisted by the application of the biocompatible particles. Accordingly, a surface micro-structure can be produced in controlled manner on the biocompatible base layer, which exactly meets the required dimensions with respect to micro- and macro-structure and which is or can be matched to the bone or tissue type in question so that optimum ingrowth of the bone into the surface layer is ensured.

A fundamental advantage in this case is that relative movement between the bone and the joint replacement implant is minimised so that substantially no abrasion particles are formed to begin with. Should abrasion particles nevertheless be formed—which possibly, in the event of very great loading of the joint, cannot be adequately prevented despite the possibility for optimised implant anchoring according to the invention—an additional significant advantage according to the invention comes into play, namely the effect that it is very difficult for abrasion particles formed in the joint to migrate along the implant because of a labyrinth of bone and porous surface. Osteolysis proceeds along the implant at a correspondingly slower rate, which results in a significantly improved service life and long-term anchoring of the joint replacement implant in the bone.

In accordance with an embodiment of the invention, the biocompatible material is applied to the virgin surface of the implant by means of a vacuum plasma spraying method. The plasma flame is so adjusted that even though the titanium particles are caused to start to melt slightly, they are only slightly compacted when they impact on the substrate, namely the virgin surface of the implant. By that means it is ensured that the surface retains pores which are open to the outside, smoothing of the applied surface is avoided and a macro-structure is produced. Vascularised bone tissue can grow into that macro-structure, which has pores having an average diameter of 300 micrometres. It should already be mentioned at this point that the diameter of the pores can be varied, as described hereinbelow, in dependence upon the nature of the starting material or the nature of the metal applied to the surface of the implant.

An alternative method of applying the biocompatible metal is brushing, spreading, spraying or any other application technique suitable for applying a flowable product or paste to an article.

A paste of such a kind is produced by mixing the biocompatible metal or an alloy thereof together with one or more binders and/or sintering adjuvant(s) and adjusting to a flowable consistency. The degree of requisite flowability depends substantially on the surface shape of the implant and the selected mode of application of the paste or liquid comprising the biocompatible metal. The finer the structures of the substrate to which the biocompatible metal is to be applied, the less viscous the paste or liquid should be, in order to be able to fill even fine structures completely, but without tending to form drops and to run. The same also applies, of course, if the biocompatible metal is applied by spraying or spattering.

In accordance with the invention, one or more of the following substances are provided as binder: carboxymethyl cellulose, collodium, polyvinyl alcohol, water or an inorganic solvent. A necessary criterion when selecting the binder is the possible of substantially completely removing it from an implant surface. Removal can be carried out during processing of the implant surface.

In accordance with a constructional variant of the invention, the at least one layer applied to the virgin surface of the implant is sintered. Sintering is advantageously used when the biocompatible metal has been applied to the virgin surface of the implant by means of brushing, spreading, spraying, spattering or any other application technique of such a kind. Sintering of a biocompatible layer applied by means of a vacuum plasma spraying method is possible, especially when the layer applied by the vacuum plasma spraying method has pores that are too large, that is to say the pore size of the implant surface can be revised by means of sintering.

The afore-mentioned sintering adjuvant is, in accordance with the invention, a sintering adjuvant metal which, together with the biocompatible metal or alloy thereof, forms a low-melting eutectic. For that purpose, silicon or cobalt, preferably in elemental form, is especially used. Usually, the silicon or cobalt is used in the form of a powder, which can be mixed with a metal powder and one or more binders so that a paste having a homogeneous distribution of constituents can be produced and also applied in homogeneous form to the virgin surface of the implant.

In accordance with an embodiment of the invention, sintering is carried out in vacuo. In that case, the advantage is that, as a result of sintering in vacuo, debindering takes place during the heating-up phase, in the course of which the binder is denatured and/or drawn out of the system. A further advantage is that, instead of a biocompatible metal, for example instead of titanium powder, it is possible to use its corresponding precursor, namely the corresponding metal hydride powder. In that case, dehydrogenation also occurs in the heating-up phase of the sintering cycle.

A sintering temperature in that case is in the range from 800° C. to 1500° C. or in the range from 950° C. to 1400° C. and especially in the range from 1000° C. to 1350° C. The suitable temperature in each particular case is crucially dependent on the sintering adjuvants used and the ratio thereof relative to the biocompatible metal, and in the case of silicon is in the range from 1295° C. to 1355° C. When cobalt is used as sintering adjuvant, a preferred sintering temperature that is used is in the range from 1000° C. to 1100° C.

In accordance with the invention, the biocompatible metal is used in powder form, especially in the form of an angular powder. Accordingly, in advantageous manner, the formation of an excessively compact implant surface can be avoided because angular particles do not allow dense packing (sphere packing) on account of their irregular structure and corners. As a result, a certain basic porosity is provided from the outset, which in terms of pore depth corresponds to the layer thickness of the surface layer on the implant.

The layer thickness of the open-pored surface layer is in the range from 0.1 mm to 2.5 mm, preferably in the range from 0.3 mm to 1.9 mm and especially in the range from 0.5 mm to 1.5 mm. Sufficient depth of contact is accordingly provided for “grab” between the implant and bone.

The biocompatible metal applied to the virgin surface of the implant advantageously has a particle size in the range from 50 μm to 800 μm, preferably in the range from 100 μm to 650 μm and especially in the range from 200 μm to 550 μm. It is accordingly possible to structure the surface in dependence on the particle size, making it possible, in combination with the angular form of a powder which has, for example, been ground so as to be angular and/or in combination with the layer thickness of the open-pored surface layer, to select an optimised pore diameter for bone to grow into. Furthermore, undercuts and cavities are possible within the layer so that bone that is growing in can engage behind the porous surface layer and, by that means, ensure optimum anchoring.

As biocompatible metal, in accordance with the invention, preferably titanium, but also zirconium, niobium or tantalum, are provided. Those metals exhibit excellent biocompatibility and allow different metals to be used should one metal give rise to an unforeseeable lack of compatibility in an individual.

Furthermore, the biocompatible metal can be used in the form of a metal hydride powder. This is advantageous because the metal hydride powder is a precursor of the actual metal in the production thereof. In the case of a powder of such a kind, dehydrogenation takes place in, for example, the heating-up phase of the sintering cycle when the latter is carried out in vacuo.

In accordance with an embodiment of the invention, the etching of the implant surface is carried out by means of an acid bath and/or by means of plasma etching. In the case of plasma etching, the use of an oxygen plasma is especially advantageous. The method to be used in a particular case will depend on the nature of the desired surface micro-structure. Whereas, an etching process in an acid bath results in the formation of small etching pits having a diameter of from 0.1 μm to 2.5 μm, preferably from 0.5 μm to 1.9 μm and especially in the order of magnitude of from 0.8 μm to 1.5 μm, plasma etching does not result in pit formation, but rather forms a microscopically fine, relatively shallow roughening of the metal surface. The two methods can be used successively in combination.

A further possible means of producing a surface micro-structure on the implant surface is the application of fine biocompatible particles to the implant surface, as already mentioned hereinbefore. The fine biocompatible particles in this case have a particle size in the range from 0.01 μm to 5 μm, preferably in the range from 0.1 μm to 3 μm, especially in the range from 0.2 μm to 1 μm. Using this method, no roughening or etching of the implant surface takes place; rather, the surface is coated with the biocompatible particles. This is accomplished, for example, using a sol-gel method and a binder, preferably a silicate-based binder. This particle binder, unlike the aforementioned binder for producing a paste or liquid, remains behind on the biocompatible particles and, as such, is likewise biocompatible. Suitable materials for the fine biocompatible particles are, especially, titanium dioxide or calcium phosphate. However, it is also possible to use another suitable biocompatible material. The two mentioned materials are, however, especially advantageous because they correspond to the body's own compounds or to compounds which are identical or similar to the implant, which are, as such, biocompatible.

The problem according to the invention is furthermore solved by an open-pored biocompatible surface layer which is arranged on a virgin surface of the implant and which has a layer thickness in the range from 0.1 mm to 2.5 mm, preferably in the range from 0.3 mm to 1.9 mm, especially in the range from 0.5 mm to 1.5 mm, and a porosity in the range from 20% to 85%, preferably in the range from 30% to 70% and especially in the range from 35% to 65%. That high degree of porosity, which can in individual cases also be greater than 85%, provides for an optimised possibility for the anchoring of the bone in the open-pored surface layer.

As a topographical stimulus for the ingrowth of bone in rapid and optimised manner, there is provided, in accordance with the invention, a surface structure in the sub-micrometre range to the micrometre range. This structure consists, on the one hand, of etching pits having a diameter in the range from 0.1 μm to 2.5 μm, preferably in the range from 0.5 μm to 1.9 μm and especially in the range from 0.8 μm to 1.5 μm, and, on the other hand, of a shallow roughening of the open-pored surface layer in the sub-micrometre range and in the micrometre range.

In accordance with a further embodiment of the invention, the implant has biocompatible particles, especially of titanium dioxide or potassium phosphate, at the implant surface. An at least partial coating with those particles is possible. In accordance with the invention, the biocompatible particles have a particle size in the range from 0.1 μm to 5 μm, preferably in the range from 0.1 μm to 3 μm and especially in the range from 0.2 μm to 1 μm. Different size ranges can be combined with one another where this is desirable. However, in that case, it should be ensured that there is no closure of pores by the biocompatible particles such that the diameter of the cavities open to the outside would fall below a size required for vascularised bone tissue to grow into.

Combinations of the various surface micro-structures, namely pits, shallow roughening and applied biocompatible particles are provided.

In accordance with a further embodiment, the open-pored surface layer of the implant consists substantially of titanium, niobium, zirconium, tantalum or alloys thereof.

Optimisation, in line with the problem, of the long-term stability of bone implant combinations is achieved by the use of a surface layer according to the invention for femoral stems, sockets for hip joints, femoral components for a knee joint replacement, tibial components for a knee joint replacement, components for a shoulder joint replacement, components for an elbow joint replacement, components for a toe joint replacement, components for a finger joint replacement, for a component for the fusion of vertebral bodies of the lumbar spine, for components for an intervertebral disc replacement, for transgingival implant systems and for orthodontic, especially jaw-orthopaedic, implant systems and tooth (replacement) implants.

Further embodiments of the invention are derived from the subordinate claims.

The invention will be described hereinbelow by means of exemplary embodiments.

Starting from a ground, angular, titanium powder produced via the hydride stage, an open-pored structure is applied to the implant surface in a first step. The titanium powder therein has a particle size of at least 200 μm. The open-pored layer itself has a layer thickness of from 0.5 to 1.5 mm and a porosity of at least 40%. The layer is preferably applied by means of the vacuum plasma spraying method. The plasma flame therein is so adjusted that even though the titanium particles are caused to start to melt slightly, they are only slightly compacted when they impact on the substrate, namely the virgin surface of the implant. The implant surface thereby produced is then subjected to an etching process in an acid bath. By appropriately controlling the process, fine etching pits having a diameter of about 1 μm are produced.

In accordance with a second exemplary embodiment, the open-pored layer is produced by means of a sintering method using a somewhat coarser, angular, titanium powder having a particle size of about 500 μm. The titanium powder is stirred up together with a binder, namely water, and a sintering adjuvant, namely elemental silicon powder, to form a spreadable paste and is applied to the virgin surface of the implant using a brush. The elemental silicon powder, together with the titanium used, forms a low-melting eutectic, which is used for temporary liquid-phase sintering. The sintering cycle itself is carried out in vacuo and includes, during heating-up, a debindering phase, in which the water is removed. Only then does the actual sintering process being. Owing to the use of silicon as sintering adjuvant, the sintering temperature is 1330° C. The open-pored layer thereby produced is then subjected to an etching process which corresponds to that of the previous example.

In accordance with a third exemplary embodiment, elemental cobalt powder is used as sintering agent instead of silicon powder. Otherwise, this example is the same as the previous, second exemplary embodiment. Owing to the use of cobalt as sintering adjuvant, the sintering temperature is about 1040° C.

In accordance with a fourth exemplary embodiment, the sintering cycle is carried out in vacuo. The precursor of titanium powder, namely the corresponding titanium hydride powder, is used as biocompatible material. In the case of this powder, dehydrogenation takes place in the heating-up phase of the sintering cycle. Otherwise, this exemplary embodiment corresponds to exemplary embodiment 2. In this case too, water is used as binder, which is likewise removed from the system in the heating-up phase of the sintering cycle.

Instead of the acid etching, plasma etching can be used for the layers produced by means of vacuum plasma spraying methods or by means of sintering. The etching is preferably carried out in oxygen plasma. No pit formation occurs in the process; rather, a microscopically fine, somewhat shallow roughening of the titanium surface is brought about.

At this point, it should be emphasised that, of course, the implant virgin surface can also be etched before application of at least one layer of the biocompatible metal. Application of a plurality of layers of the biocompatible metal is also possible and will depend primarily on the desired layer thickness and desired structuring of the porous surface layer and on the form of the pores. It is accordingly possible, for example, for a gradient in terms of pore diameter to be produced so that the pores become narrower from the outer surface towards the implant virgin surface; it is likewise possible for the pore diameter to become wider towards the implant virgin surface. By that means it is advantageously possible for an anchor-like collection of osteoblasts to be formed with a connection to the bone inside the porous surface layer. The porous surface layer would therefore be undercut in the direction of the implant virgin surface so that an anchoring action of the bone at the porous surface layer is optimised.

In accordance with a fifth exemplary embodiment, the layer produced by means of vacuum plasma spraying methods or by means of sintering is not etched, but, rather, coated with fine biocompatible particles. In two variants, either titanium dioxide powder or calcium phosphate powder is used as those fine particles. The particle size in both cases is 1 μm. The powders are applied in a sol-gel method using a silicate-based binder.

At this point, it should be pointed out that all the above-mentioned parts are claimed for themselves alone and in any combination as being of inventive significance. Modifications thereof are within the purview of the person skilled in the art.

1. An open-pored biocompatible surface layer for an implant, which layer is arranged on a virgin surface of the implant, comprising:
an open-pored surface layer with a thickness in a range selected from the group consisting of the range from 0.1 mm to 2.5 mm inclusive, the range from 0.3 mm to 1.9 mm inclusive, and the range from 0.5 mm to 1.5 mm inclusive; and
the porosity of the open-pored surface layer is in a range selected from the group consisting of the range from 20% to 85% inclusive, the range from 30% to 70% inclusive, and the range from 35% to 65% inclusive.

2. The surface layer according to claim 1, wherein
the open-pored surface layer has pits or etching pits, having a diameter in a range selected from the group consisting of the range from 0.1 μm to 2.5 μm inclusive, the range from 0.5 μm to 1.9 μm inclusive, and the range from 0.8 μm to 1.5 μm inclusive.

3. The surface layer according to claim 1, wherein
the open-pored surface layer has a shallow roughening in the sub-micrometer range.

4. The surface layer according to claim 1, further comprising particles arranged on the implant surface, said particles selected from the group consisting of
biocompatible particles, titanium dioxide biocompatible partiecles, and calcium phosphate biocompatible particles.

5. The surface layer according to claim 4, wherein
the biocompatible particles have a particle size in a range selected from the group consisting of the range from 0.01 μm to 5 μm inclusive, the range from 0.1 μm and 3 μm inclusive, and the range from 0.2 μm to 1 μm inclusive.

6. The surface layer according to claim 1, wherein
the open-pored surface layer consists substantially of a material selected from the group consisting of titanium, zirconium, niobium or tantalum.

7. The surface layer according to claim 1, wherein
the open-pored surface layer is sintered.

8. A method of producing an implant selected from the group consisting of an open-pored coated implant, and a joint replacement implant, comprising:
applying least one layer of a biocompatible metal or an alloy thereof to a virgin surface of the implant, to produce an implant surface; and
producing a surface micro-structure on the implant surface by means selected from the group consisting of etching of the implant surface, application of fine biocompatible particles to the implant surface, and etching of the implant surface and application of fine biocompatible particles to the implant surface.

9. The method according to claim 8, wherein
the biocompatible metal is applied by means of a vacuum plasma spraying method.

10. The method according to claim 8, wherein
the biocompatible metal is applied by a technique selected from the group consisting of brushing, spreading, spraying, and a like application techniques.

11. The method according to claim 8 wherein
the at least one layer applied to the virgin surface of the implant is sintered.

12. The method according to claim 11, wherein
Materials selected from the group consisting of binders, sintering adjuvants, and binders and sintering adjuvants are used.

13. The method according to claim 12, wherein
as sintering adjuvant there is used a sintering adjuvant metal which, together with the biocompatible metal or alloy thereof, forms a eutectic selected from the group consisting of low-melting eutectic, silicon, cobalt, and a eutectic in elemental powder form.

14. The method according to claim 11 wherein
sintering is carried out in vacuo.

15. The method according to claim 11 wherein
sintering comprises a phase selected from the group consisting of a debindering phase, a dehydrogenation phase, and a debindering and dehydrogenation phase.

16. The method according to claim 11 wherein
a sintering temperature in a range selected from the group consisting of the range from 800° C. to 1500° C. inclusive, the range from 950° C. to 1400° C. inclusive, and the range from 1000° C. to 1350° C. inclusive is used.

17. The method according to claim 8 wherein
the biocompatible metal is used in a form selected from the group consisting of powder form and an angular powder.

18. The method according to claim 8 wherein
a layer thickness of the open-pored surface layer in a range selected from the group consisting of the range from 0.1 mm to 2.5 mm inclusive, the range from 0.3 mm to 1.9 mm inclusive, and the range from 0.5 mm to 1.5 mm is produced.

19. The method according to claim 8 wherein
the biocompatible metal applied to the virgin surface of the implant has a particle size in a range selected from the group consisting of the range from 50 μm to 800 μm inclusive, the range from 100 μm to 650 μm inclusive, and the range from 200 μm to 550 μm inclusive.

20. The method according to claim 8 wherein
the biocompatible metal is selected from the group consisting of titanium, zirconium, niobium, and tantalum.

21. The method according to claim 8, wherein
the biocompatible metal is used in the form of a metal hydride powder.

22. The method according to claim 8, wherein
the etching of the implant surface is carried out by means of a technique selected from the group consisting of acid (bath) etching, plasma etching, oxygen plasma etching, acid (bath) etching and plasma etching, and acid (bath) etching and oxygen plasma etching.

23. The method according to claim 8, wherein
the fine biocompatible particles have a particle size in a range selected from the group consisting of the range from 0.01 μm to 5 μm inclusive, the range from 0.1 μm to 3 μm inclusive, and the range from 0.2 μm to 1 μm inclusive.

24. The method according to claim 8, wherein
the fine biocompatible particles are applied by a sol-gel method using a binder selected from the group consisting of a binder and a silicate-based binder.

25. The method according to claim 8, wherein
A material selected from the group consisting of titanium dioxide, calcium phosphate, and another biocompatible material is used as material for the fine biocompatible particles.

26. An implant having a surface layer according to claim 1.

27. Use of a surface layer according to claim 1 for an implant selected from the group consisting of femoral stems, a sockets for a hip joints, a femoral components for a knee joint replacement, a tibial components for a knee joint replacement, a components for a shoulder joint replacement, a components for an elbow joint replacement, a components for a toe joint replacement, a components for a finger joint replacement, a component for the fusion of vertebral bodies of the lumbar spine, a components for an intervertebral disc replacement, a transgingival implant systems, an orthodontic implant systems and a tooth (replacement) implants.

28. The implant according to claim 26, wherein the implant is a joint replacement implant.
